(19) United States
(12) Patent Application Publication
(10) Pub. No.: US 2007/0195550 A1
(43) Pub. Date: Aug. 23, 2007

(54) FOLDABLE ELECTRONIC DEVICE

(75) Inventor: Chih-Sheng Tsai, Taipei (TW)

Correspondence Address: JIANQ CHYUN INTELLECTUAL PROPERTY OFFICE, 7 FLOOR-1, NO. 100, ROOSEVELT ROAD, SECTION 2, TAIPEI, 100, TW

(73) Assignee: ASUSTeK COMPUTER INC., Taipei (TW)

(21) Appl. No.: 11/469,463

(22) Filed: Aug. 31, 2006

(30) Foreign Application Priority Data
Sep. 5, 2005 (TW) 94130269

(51) Int. Cl. F21V 7/04

(52) U.S. Cl. 362/600

(57) ABSTRACT

A foldable electronic device including a main frame, a display and a pivot is provided. The main frame includes a photo catalyst disposed on the surface thereof The display includes an ultraviolet light source for illuminating the photo catalyst disposed on the main frame. The main frame is pivoted on one side of the display through a pivot, so that the display can rotate relatively to the main frame. When the main frame and the display are closed, the photo catalyst is exposed to ultraviolet light generated from the ultraviolet light source for sterilization.

### CROSS-REFERENCE TO RELATED APPLICATION

This application claims the priority benefit of Taiwan application serial no. 94130269, filed Sep. 5, 2005. All disclosure of the Taiwan application is incorporated herein by reference.

### BACKGROUND OF THE INVENTION

1. Field of the Invention

The present invention generally relates to a foldable electronic device, and particularly to a foldable electronic device having sterilization function.

2. Description of Related Art

To meet the requirements of high-speed, high-efficiency, lightweight and slimness of modem electronic products, electronic components are increasingly miniaturized. Hence, foldable electronic devices have become the mainstream in the modern life. These foldable electronic devices include notebooks, mobile phones, electronic dictionaries, personal digital assistants (PDAs), web pads and so on.

These foldable electronic devices provide users with much convenience. Under the condition that users use these devices frequently, germs propagate on the elements of these devices, such as keyboards, touch pads and so on, due to the touches by the users, pollution or other factors. These germs are harmful to human health. The propagation of the germs is like a timeless bomb that threatens a user's health at any moment. In prior art, a photo catalyst is added to these elements which are easily to accumulate germs; through the exposure of light, the bacteria can be killed.

It should be noted that the photo catalyst will have a better sterilization result when exposed under ultraviolet light. However, the photo catalyst is generally exposed to visible light having longer wavelength, which is emitted from fluorescent lamps or inner parts of the foldable electronic devices. In fact, these lights can not effectively drive the photo catalyst to perform sterilization function. As a result, even if the photo catalyst is added to these elements where germs are easily accumulated, it is still not able to eliminate the germs efficiently.

### SUMMARY OF THE INVENTION

Accordingly, the present invention is directed to a foldable electronic device having a sterilization function.

The present invention is directed to a foldable electronic device which comprises a main frame, a display and a pivot. A photo catalyst is disposed on a surface of the main frame. The display comprises an ultraviolet light source for illuminating the photo catalyst disposed on the main frame. The pivot is disposed at one side of the main frame and the display, such that the display can rotate relatively to the main frame.

### BRIEF DESCRIPTION OF THE DRAWINGS

The accompanying drawings are included to provide a further understanding of the invention, and are incorporated in and constitute a part of this specification. The drawings illustrate embodiments of the invention and, together with the description, serve to explain the principles of the invention.

FIG. 1 illustrates a schematic view showing a foldable electronic device according to an embodiment of the present invention.

FIG. 2 illustrates a schematic view showing the display shown in FIG. 1.

FIG. 3 illustrates a schematic view showing the backlight module shown in FIG. 2.

FIG. 4 illustrates a schematic view showing the foldable electronic device when the ultraviolet light source is turned on.

FIG. 5 illustrates a schematic view showing another display.

FIG. 6 illustrates a schematic view showing another backlight module.

FIG. 7 illustrates a schematic view showing the foldable electronic device during an ultraviolet sterilization process.

### DESCRIPTION OF THE EMBODIMENTS

Reference will now be made in detail to the present preferred embodiments of the invention, examples of which are illustrated in the accompanying drawings. Wherever possible, the same reference numbers are used in the drawings and the description to refer to the same or like parts.

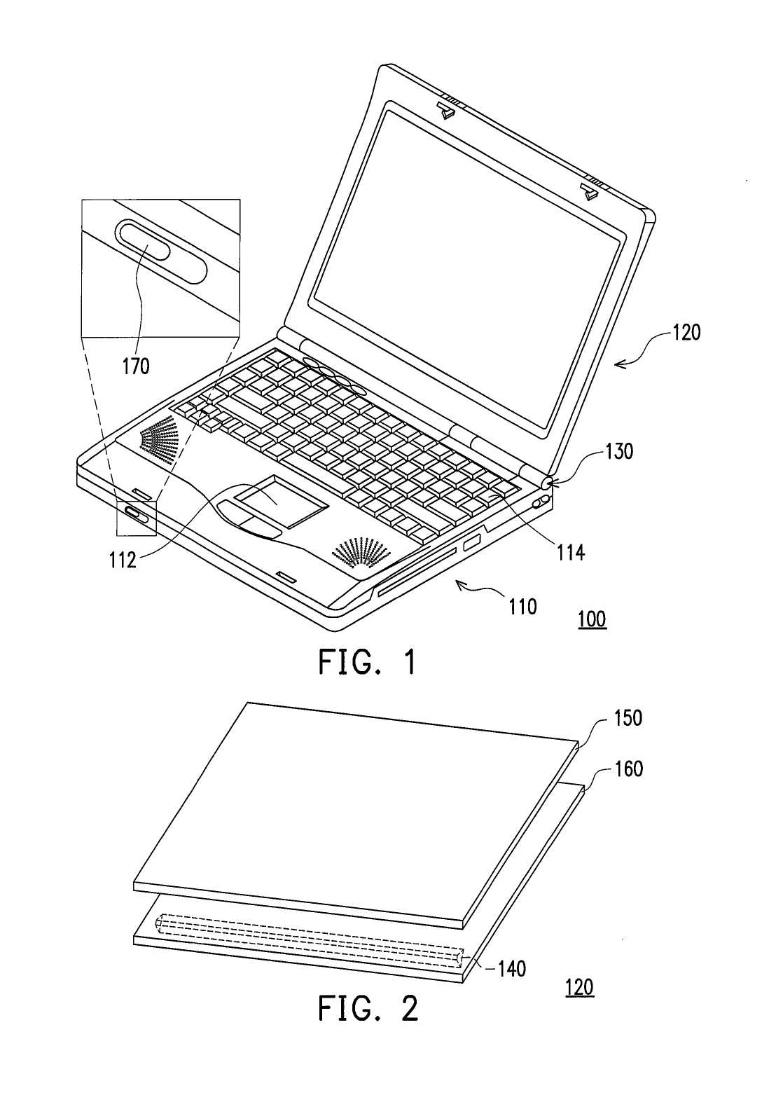

FIG. 1 illustrates a schematic view showing a foldable electronic device according to an embodiment of the present invention. FIG. 2 illustrates a schematic view showing the display shown in FIG. 1. Please refer to FIGS. 1 and 2, the foldable electronic device 100 mainly comprises a main frame 110, a display 120 and a pivot 130. The pivot 130 is disposed at one side of the main frame 110 and the display 120, such that the display 120 can rotate relatively to the main frame 110. A photo catalyst is disposed on a surface of the main frame 110, and the material of the photo catalyst comprises titanium dioxide. It should be noted that the foldable electronic device 110 is a notebook computer in this embodiment, the main frame 110 comprises a touch pad 112 and a keyboard 114, and the photo catalyst is disposed on the touch pad 112 and the keyboard 114. However, the position of the photo catalyst is not limited in the present invention. In other embodiment, the main frame 110 of the foldable electronic device may only comprise the touch pad 112 and the keyboard 114, and the photo catalyst is disposed on the touch pad 112 or the keyboard 114, for example.

The display 120 of this embodiment comprises an ultraviolet light source 140 for illuminating the photo catalyst disposed on the main frame 110. The ultraviolet light source 140 may comprise an ultraviolet tube. Practically, the display 130 mainly comprises a display panel 150 and a first backlight module 160. The display panel 150 is, for example, a liquid crystal display panel, and the first backlight module 160 is disposed on one side of the display panel 150. Besides, the ultraviolet light source 140 is disposed within the first backlight module 160.

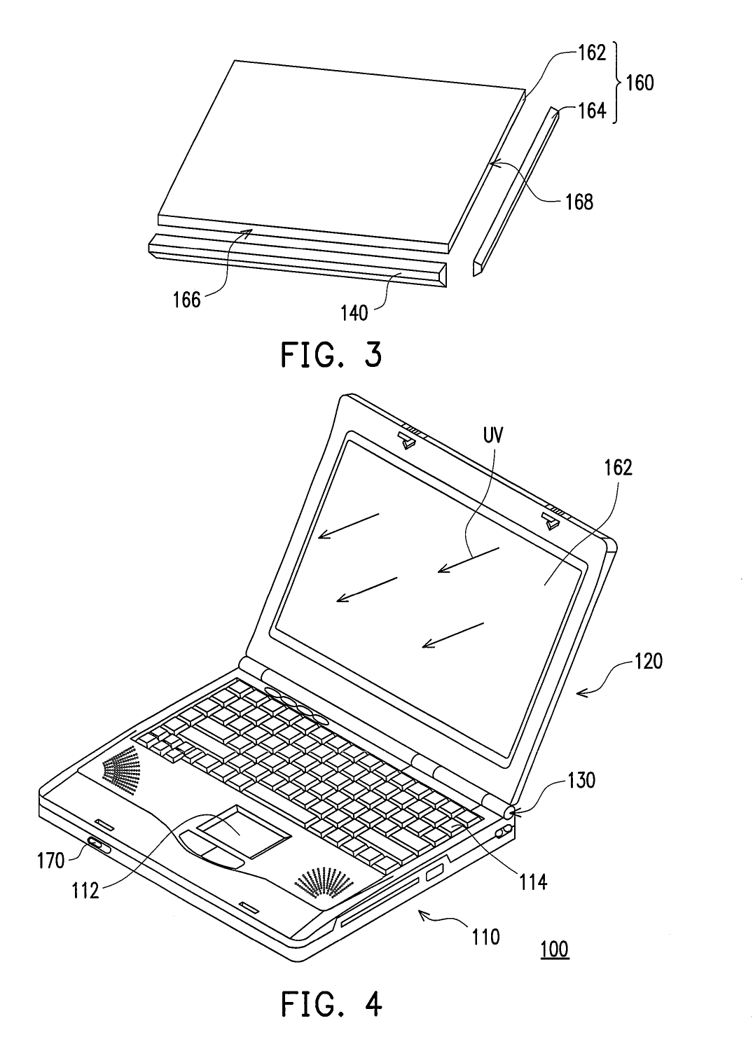

FIG. 3 illustrates a schematic view showing the backlight module shown in FIG. 2. Please refer to FIG. 3, the first backlight module 160, for example, comprises a first light-guiding plate 162, a white light source 164 and the above-mentioned ultraviolet light source 140. The first light-guiding plate 162 has a first light incident surface 166 and a second light incident surface 168. The ultraviolet light source 140 is disposed beside the first light incident surface 166. The white light source 164 is disposed beside the second light incident surface 168 to provide light which the display panel 150 needs. The white light source 164 can be a cold cathode fluorescent lamp, a light emitting diode array or other form of light source.

In accordance with the above description, when the ultraviolet light source 140 is turned on, the ultraviolet light generated from the ultraviolet light source 140 travels through the display panel 150 via the first light-guiding plate 162 and illuminates the photo catalyst disposed on the main frame 110 as shown in FIG. 4. The result of sterilization is at its best when the photo catalyst is exposed to the ultraviolet light. Therefore, the foldable electronic device 100 can eliminate germs propagated on the main frame 110 (such as the surface of the touch panel 112 and the keyboard 114) efficiently by this design.

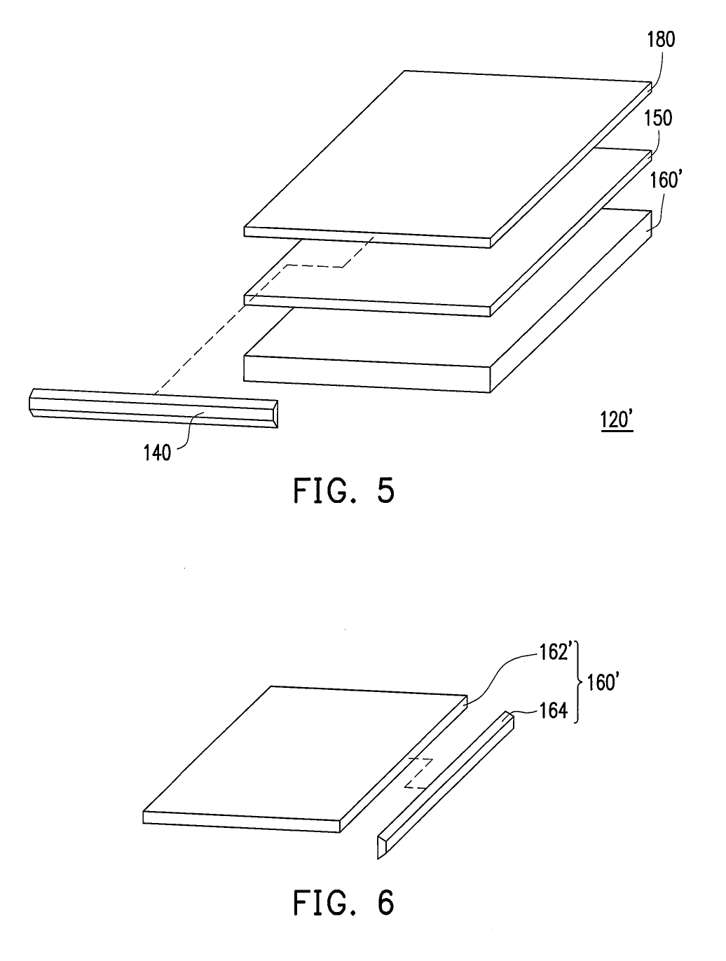

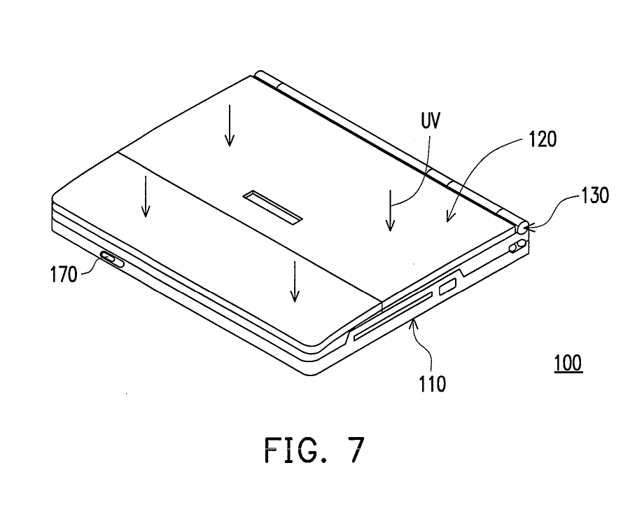

Except the arrangement of the display 120 of the foldable electronic device shown in FIG. 2, the display can have other arrangement as shown in FIG. 5. FIG. 6 illustrates a schematic view showing a second backlight module as shown in FIG. 5. Please refer to FIGS. 5 and 6, the display 120′ comprises a display panel 150, a second backlight module 160′ and a second light-guiding plate 180. The second backlight module 160′ is disposed on one side of the display panel 150. The second light-guiding plate 180 is disposed on the other side of the display panel 150 for protecting the display panel 150; the ultraviolet light source 140 is disposed beside the second light-guiding plate 180. The second backlight module 160′ mainly comprises a third light-guiding plate 162′, the above-mentioned ultraviolet light source 140 and a white light source 164. The white light source 164 is disposed beside the third light-guiding plate 162′ for providing light which the display panel 162′ needs.

The same reference numbers are used in the display 120′ and 120, and the description to refer to the same or like parts, and therefore the display 120′ is not repeated herein. The difference between the displays 120 and 120′ is that when the ultraviolet light source 140 is turned on, the ultraviolet light generated from the ultraviolet light source 140 travels through the second light-guiding plate 180 directly and illuminates the photo catalyst disposed on the main frame 110. Although the structure between the displays 120 and 120′ is different, the illumination effect of the ultraviolet light is still the same.

Since ultraviolet light is harmful to humans, people should avoid being exposed to ultraviolet light. To avoid the ultraviolet light emitted from the display panel 150 when the ultraviolet light source 140 of the display 120 is turned on, the ultraviolet light source 140 of the foldable electronic device 100 will only be turned on when the foldable electronic device 100 is closed.

For example, the foldable electronic device 100 of the present invention comprises an ultraviolet light source switch 170 (as shown in FIG. 1) to switch the ultraviolet light source 140 on or off. When sterilization is to be performed, the foldable electronic device 100 will be closed first; then the ultraviolet light source 140 is turned on by using the ultraviolet light source switch 170 so as to expose the ultraviolet light to the photo catalyst. It should be noted that the ultraviolet light source 140 only emits ultraviolet light when the main frame 110 and the display 120 of the foldable electronic device 100 are closed and the ultraviolet light source switch 170 is turned on. When the display 120 is opened, this design of the foldable electronic device 100 can prevent users from being exposed to ultraviolet light if the ultraviolet light source switch 170 is switched accidentally. One of ordinary skill in the art will understand that the ultraviolet light source 140 can emit ultraviolet light by either manually or automatically when the main frame 110 and the display 120 are closed.

The present invention utilizes the ultraviolet light source disposed within the foldable electronic device to illuminate the photo catalyst disposed on the main frame. The photo catalyst exposed to ultraviolet light having short wavelength can achieve its optimum sterilization effect. Accordingly, the foldable electronic device of the present invention having ultraviolet light source can further eliminate germs propagated on the surface of the main frame. Furthermore, the ultraviolet light source of the present invention only emits ultraviolet light when the main frame and the display of the foldable electronic device are closed so as to prevent users from being exposed to ultraviolet light.

It will be apparent to those skilled in the art that various modifications and variations can be made to the structure of the present invention without departing from the scope or spirit of the invention. In view of the foregoing, it is intended that the present invention cover modifications and variations of this invention provided they fall within the scope of the following claims and their equivalents.

What is claimed is:

1. A foldable electronic device, comprising:
a main frame, a photo catalyst being disposed on a surface of the main frame;
a display comprising an ultraviolet light source for illuminating the photo catalyst disposed on the main frame; and
a pivot disposed at one side of the main frame and the display, such that the display can rotate relatively to the main frame.

2. The foldable electronic device according to claim 1, wherein the display comprises:
a display panel; and
a first backlight module disposed on one side of the display panel, and the ultraviolet light source disposed in the first backlight module.

3. The foldable electronic device according to claim 2, wherein the first backlight module comprises:
a first light-guiding plate having a first light incident surface and a second light incident surface, wherein the ultraviolet light source is disposed beside the first light incident surface; and
a white light source disposed beside the second light incident surface for providing light which the display panel needs.

4. The foldable electronic device according to claim 3, wherein the white light source comprises a cold cathode fluorescent lamp or a light emitting diode array.

5. The foldable electronic device according to claim 1, wherein the display comprises:
a display panel;
a second backlight module disposed at one side of the display panel; and
a second light-guiding plate disposed at the other side of the display panel, wherein the ultraviolet light source is disposed beside the second light-guiding plate.

6. The foldable electronic device according to claim 5, wherein the second backlight module comprises:
a third light-guiding plate; and
a white light source disposed beside the third light-guiding plate for providing light which the display panel needs.

7. The foldable electronic device according to claim 6, wherein the white light source comprises a cold cathode fluorescent lamp or a light emitting diode array.

8. The foldable electronic device according to claim 1, wherein the material of the photo catalyst comprises titanium dioxide.

9. The foldable electronic device according to claim 1, wherein the main frame further comprises an ultraviolet light source switch, when the foldable electronic device is closed and the ultraviolet light source switch is turned on, the ultraviolet light source switch is suitable for emitting an ultraviolet light.

10. The foldable electronic device according to claim 1, wherein the ultraviolet light source comprises an ultraviolet tube.

11. The foldable electronic device according to claim 1, wherein the main frame further comprises a touch panel and/or a keyboard, and the photo catalyst is disposed on the surface of the touch panel and/or the keyboard.
